(19) United States
(12) Patent Application Publication
(10) Pub. No.: US 2019/0038895 A1
(43) Pub. Date: Feb. 7, 2019

(54) SYSTEMS AND METHODS FOR MAKING AND USING ELECTRICAL STIMULATION AND RF ABLATION DEVICES WITH ELECTROMAGNETIC NAVIGATION

(71) Applicant: Boston Scientific Neuromodulation Corporation, Valencia, CA (US)

(72) Inventors: Anne Margaret Pianca, Santa Monica, CA (US); Rafaei Carbunaru, Valley Village, CA (US)

(21) Appl. No.: 16/049,498

(22) Filed: Jul. 30, 2018

Related U.S. Application Data
(60) Provisional application No. 62/541,300, filed on Aug. 4, 2017.

(51) Int. Cl. A61N 1/06; A61B 18/14; A61B 5/06; A61N 1/05

(52) U.S. Cl. CPC A61N 1/06; A61B 18/1492; A61N 1/0553; A61N 1/0558; A61B 5/065

(57) ABSTRACT

An electrical stimulation lead includes: a lead body having a proximal portion, a distal portion, and a longitudinal length; electrodes disposed along the distal portion of the lead body; terminals disposed along the proximal portion of the lead body; and conductors electrically coupling the terminals to the electrodes. A sensor assembly is disposed in the lead body in proximity to the electrodes. The sensor assembly is configured and arranged for sensing a magnetic field in three different directions to facilitate determination of a position and orientation of the electrodes based on the sensing. The sensor assembly has a largest transverse dimension of no greater than 0.5 millimeters.

### CROSS-REFERENCE TO RELATED APPLICATIONS

This application claims the benefit under 35 U.S.C. § 119(e) of U.S. Provisional Patent Application Ser. No. 62/541,300, filed Aug. 4, 2017, which is incorporated herein by reference.

### FIELD

The present invention is directed to the area of electrical stimulation systems and RF ablation systems and methods of using the systems. The present invention is also directed to systems and methods for using an electromagnetic tracking system to facilitate navigational guidance of electrostimulation leads and RF ablation systems within patients.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include an implantable pulse generator (“IPG”), one or more leads, and an array of stimulator electrodes on each lead. The stimulator electrodes are in contact with or near the nerves, muscles, or other tissue to be stimulated. The pulse generator generates electrical pulses that are delivered by the electrodes to body tissue.

### BRIEF SUMMARY

In one embodiment, an electrical stimulation lead includes a lead body having a proximal portion, a distal portion, and a longitudinal length; electrodes disposed along the distal portion of the lead body; terminals disposed along the proximal portion of the lead body; and conductors electrically coupling the terminals to the electrodes. A sensor assembly is disposed in the lead body in proximity to the electrodes. The sensor assembly is configured and arranged for sensing a magnetic field in three different directions to facilitate determination of a position and orientation of the plurality of electrodes based on the sensing. The sensor assembly has a largest transverse dimension of no greater than 0.5 millimeters.

In at least some embodiments, the sensor assembly is aligned with at least one of the electrodes along the longitudinal length of the lead body. In at least some embodiments, the electrical stimulation lead further includes a distal tip, and the sensor assembly is disposed along the distal portion of the lead between the electrodes and the distal tip.

In at least some embodiments, the lead body defines a central lumen extending along the longitudinal length of the lead body, and the sensor assembly is disposed in the central lumen.

In at least some embodiments, the lead body defines conductor lumens extending along the longitudinal length of the lead body, where the conductors are disposed in the conductor lumens, and where the sensor assembly is also disposed in one of the conductor lumens. In at least some embodiments, the sensor assembly is disposed in one of the conductor lumens distally from the at least one conductor which is also disposed in that conductor lumen. In at least some embodiments, the electrodes include a proximal-most electrode along the longitudinal length of the lead body; the conductors include a first conductor, the conductor lumens include a first conductor lumen; the first conductor is disposed in the first conductor lumen and is electrically coupled to the proximal-most electrode; and the sensor assembly is disposed in the first conductor lumen distally from the first conductor along the longitudinal length of the lead body.

In another embodiment, an electrical stimulation system includes the electrical stimulation lead described above; and a control module coupleable to the electrical stimulation lead. The control module includes a housing, and an electronic subassembly disposed in the housing. In at least some embodiments, the electrical stimulation system further includes a controller configured and arranged to control one or more magnetic fields sensed by the sensor assembly and to determine the position and orientation of the sensor assembly with respect to the one or more magnetic fields based on received signals output from the sensor assembly. In at least some embodiments, the electrical stimulation system further includes a magnetic field generator configured and arranged for generating the one or more magnetic fields sensed by the sensor assembly and controlled by the controller.

In yet another embodiment, a medical device kit includes an elongated member configured and arranged for at least partially introducing into a patient. A cannula is configured and arranged to receive the elongated member and to facilitate introduction of the elongated member into the patient. A stylet is configured and arranged to insert into the elongated member during introduction of the elongated member into the patient and to facilitate introduction of the elongated member into the patient. A sensor assembly is disposed on or in at least one of the cannula or the stylet. The sensor assembly is configured and arranged for sensing a magnetic field in three different directions to facilitate determination of a position and orientation of a portion of the elongated member based on the sensing. The sensor assembly has a largest transverse dimension of no greater than 0.5 millimeters.

In at least some embodiments, the elongated member is an electrical stimulation lead. In at least some embodiments, the elongated member is a radiofrequency ablation catheter. In at least some embodiments, the sensor assembly is a first sensor assembly, and wherein the medical device kit includes a second sensor assembly disposed in the elongated member.

In still yet another embodiment, a radiofrequency ablation system includes a thermocouple electrode configured and arranged for insertion into a patient and for conducting radiofrequency current to patient tissue. A sensor assembly is disposed along the thermocouple electrode. The sensor assembly is configured and arranged for sensing a magnetic field in three different directions to facilitate determination of a position and orientation of the thermocouple electrode based on the sensing. The sensor assembly has a largest transverse dimension of no greater than 0.5 millimeters.

In another embodiment, a method of placing an electrical stimulation lead at a target stimulation location within a patient includes providing the electrical stimulation described above. The electrodes of the electrical stimulation lead are advanced to, or in proximity to, the target stimulation location. The sensor assembly of the electrical stimulation lead is used to facilitate advancement of the electrodes to, or in proximity to, the target stimulation location.

In at least some embodiments, using the sensor assembly of the electrical stimulation lead to facilitate advancement of the electrodes includes disposing the sensor assembly in a conductor lumen defined in the lead body of the electrical stimulation lead. In at least some embodiments, using the sensor assembly of the electrical stimulation lead to facilitate advancement of the electrodes includes disposing the sensor assembly in a central lumen defined in the lead body of the electrical stimulation lead. In at least some embodiments, using the sensor assembly of the electrical stimulation lead to facilitate advancement of the plurality of electrodes includes positioning the sensor assembly along the lead body of the electrical stimulation lead with the sensor assembly aligned with at least one of the electrodes along the longitudinal length of the lead body. In at least some embodiments, using the sensor assembly of the electrical stimulation lead to facilitate advancement of the electrodes includes positioning the sensor assembly along the lead body of the electrical stimulation lead with the sensor assembly at least partially disposed between the electrodes and a distal tip of the electrical stimulation lead.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of an electrical stimulation system that includes a paddle body coupled to a control module via lead bodies, according to the invention;

FIG. 2 is a schematic side view of another embodiment of an electrical stimulation system that includes a percutaneous lead body coupled to a control module via a lead body, according to the invention;

FIG. 3A is a schematic side view of one embodiment of a plurality of connector assemblies disposed in the control module of FIG. 1, the connector assemblies configured and arranged to receive the proximal portions of the lead bodies of FIG. 1, according to the invention;

FIG. 3B is a schematic side view of one embodiment of a connector assembly disposed in the control module of FIG. 2, the connector assembly configured and arranged to receive the proximal portion of one of the lead body of FIG. 2, according to the invention;

FIG. 3C is a schematic side view of one embodiment of a proximal portion of the lead body of FIG. 2, a lead extension, and the control module of FIG. 2, the lead extension configured and arranged to couple the lead body to the control module, according to the invention;

FIG. 4 is a schematic overview of one embodiment of a tracking system suitable for use with the electrical stimulation systems of FIGS. 1 and 2, the tracking system including a sensor assembly suitable for insertion into an elongated medical device suitable for insertion into a patient, according to the invention;

FIG. 5 is a schematic illustration of one embodiment of a system for practicing the invention;

FIG. 6A is a schematic transverse cross-sectional view of one embodiment of a lead body that defines a central lumen and multiple conductor lumens, according to the invention;

FIG. 6B is a schematic transverse cross-sectional view of one embodiment of the lead body of FIG. 6A with conductors disposed in the conductor lumens of the lead body, according to the invention;

FIG. 7 is a schematic side view of one embodiment of electrodes disposed along a portion of a lead that defines a central lumen and conductor lumens housing conductors coupled to the electrodes, according to the invention;

FIG. 8A is a schematic side view of one embodiment of the sensor assembly of FIG. 4 disposed in the lead of FIG. 7 within one of the conductor lumens of the lead, according to the invention;

FIG. 8B is a schematic side view of one embodiment of the sensor assembly of FIG. 4 disposed in the lead of FIG. 7 within the central lumen of the lead, according to the invention;

FIG. 9A is a schematic side view of one embodiment of the sensor assembly of FIG. 4 disposed on or in a distal portion of a stylet, according to the invention;

FIG. 9B is a schematic side view of one embodiment of the stylet and sensor assembly of FIG. 9A disposed in the lead of FIG. 7 within the central lumen of the lead, according to the invention;

FIG. 10A is a schematic side view of one embodiment of the sensor assembly of FIG. 4 disposed on or in a distal portion of a lead introducer, according to the invention;

FIG. 10B is a schematic side view of one embodiment of the lead introducer and sensor assembly of FIG. 10A, the lead introducer receiving the lead of FIG. 7, according to the invention;

FIG. 11 is a schematic overview of one embodiment of components of a stimulation system, including an electronic subassembly disposed within a control module, according to the invention; and

FIG. 12 is a schematic side view of one embodiment of a radiofrequency ablation system that includes a thermocouple electrode inserted into a cannula and the sensor assembly of FIG. 4, according to the invention.

### DETAILED DESCRIPTION

The present invention is directed to the area of electrical stimulation systems and RF ablation systems and methods of using the systems. The present invention is also directed to systems and methods for using an electromagnetic tracking system to facilitate navigational guidance of electrostimulation leads and RF ablation systems within patients.

Suitable implantable electrical stimulation systems include, but are not limited to, a least one lead with one or more electrodes disposed on a distal end of the lead and one or more terminals disposed on one or more proximal ends of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference. In the discussion below, a percutaneous lead will be exemplified, but it will be understood that the methods and systems described herein are also applicable to paddle leads and other leads.

A percutaneous lead for electrical stimulation (for example, deep brain, spinal cord, peripheral nerve, or cardiac-tissue stimulation) includes stimulation electrodes that can be ring electrodes, segmented electrodes that extend only partially around the circumference of the lead, or any other type of electrode, or any combination thereof. The segmented electrodes can be provided in sets of electrodes, with each set having electrodes circumferentially distributed about the lead at a particular longitudinal position. A set of segmented electrodes can include any suitable number of electrodes including, for example, two, three, four, or more electrodes. For illustrative purposes, the leads are described herein relative to use for deep brain stimulation, but it will be understood that any of the leads can be used for applications other than deep brain stimulation, including spinal cord stimulation, peripheral nerve stimulation, dorsal root ganglion stimulation, sacral nerve stimulation, or stimulation of other nerves, muscles, and tissues.

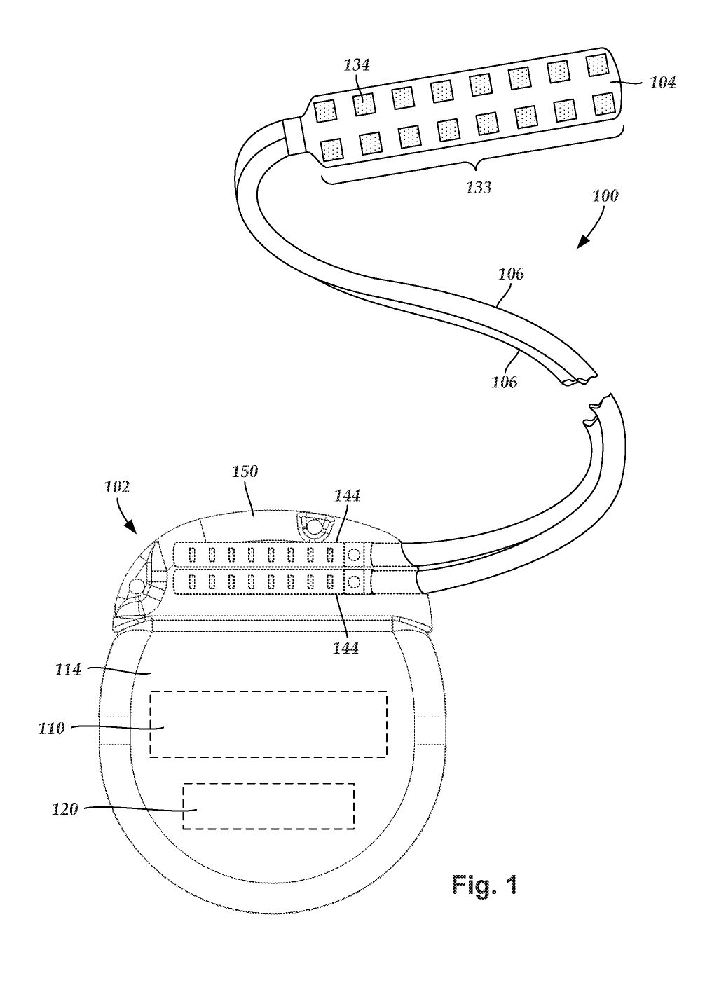

FIG. 1 illustrates schematically one embodiment of an electrical stimulation system 100. The electrical stimulation system includes a control module (e.g., a stimulator or pulse generator) 102, a paddle body 104, and one or more lead bodies 106 coupling the control module 102 to the paddle body 104. The paddle body 104 and the one or more lead bodies 106 form a lead. The paddle body 104 typically includes a plurality of electrodes 134 that form an array of electrodes 133. The control module 102 typically includes an electronic subassembly 110 and an optional power source 120 disposed in a sealed housing 114. In FIG. 1, two lead bodies 106 are shown coupled to the control module 102.

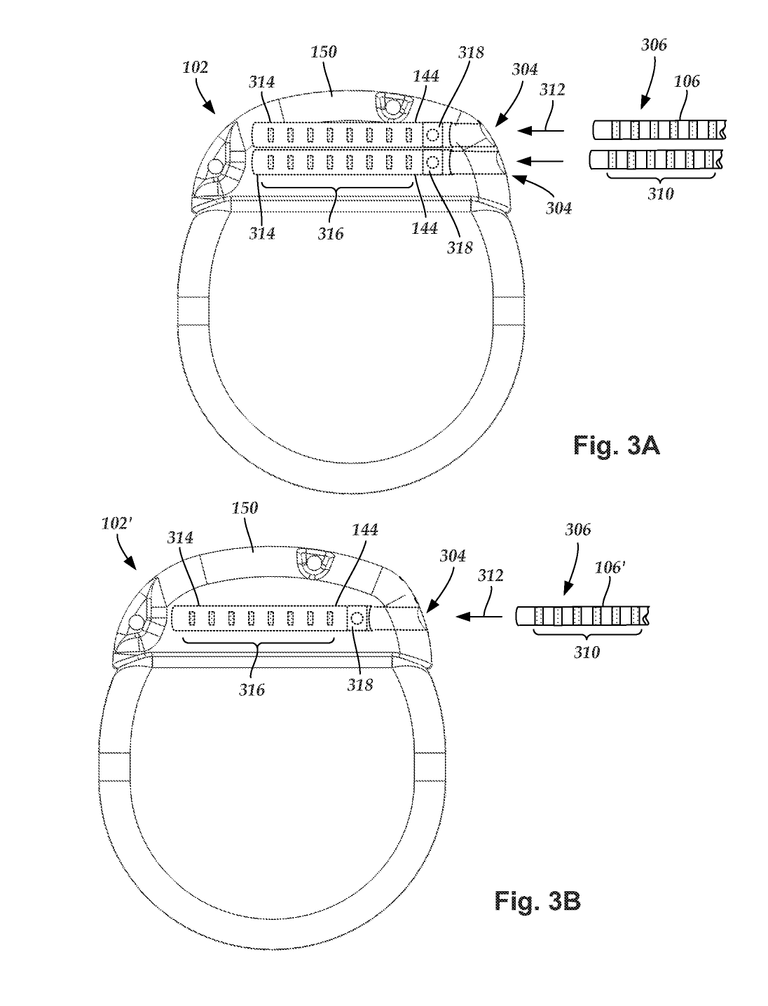

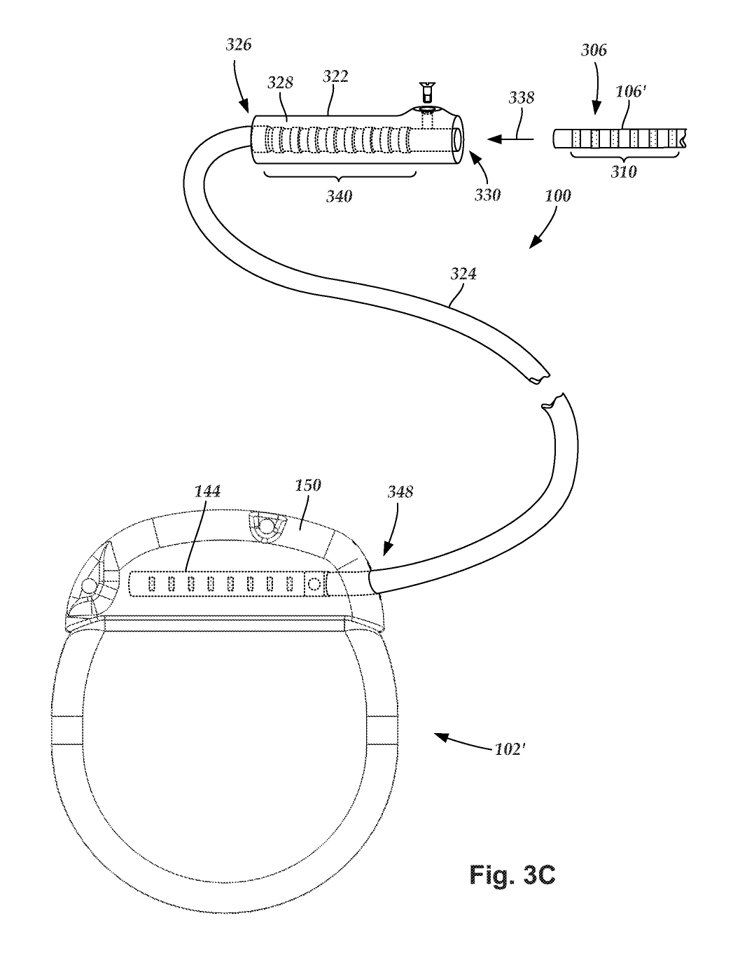

The control module 102 typically includes one or more connector assemblies 144 into which the proximal end of the one or more lead bodies 106 can be plugged to make an electrical connection via connector contacts (e.g., 316 in FIG. 3A) disposed in the connector assembly 144 and terminals (e.g., 310 in FIG. 3A) on each of the one or more lead bodies 106. The connector contacts are coupled to the electronic subassembly 110 and the terminals are coupled to the electrodes 134. In FIG. 1, two connector assemblies 144 are shown.

The one or more connector assemblies 144 may be disposed in a header 150. The header 150 provides a protective covering over the one or more connector assemblies 144. The header 150 may be formed using any suitable process including, for example, casting, molding (including injection molding), and the like. In addition, one or more lead extensions 324 (see FIG. 3C) can be disposed between the one or more lead bodies 106 and the control module 102 to extend the distance between the one or more lead bodies 106 and the control module 102.

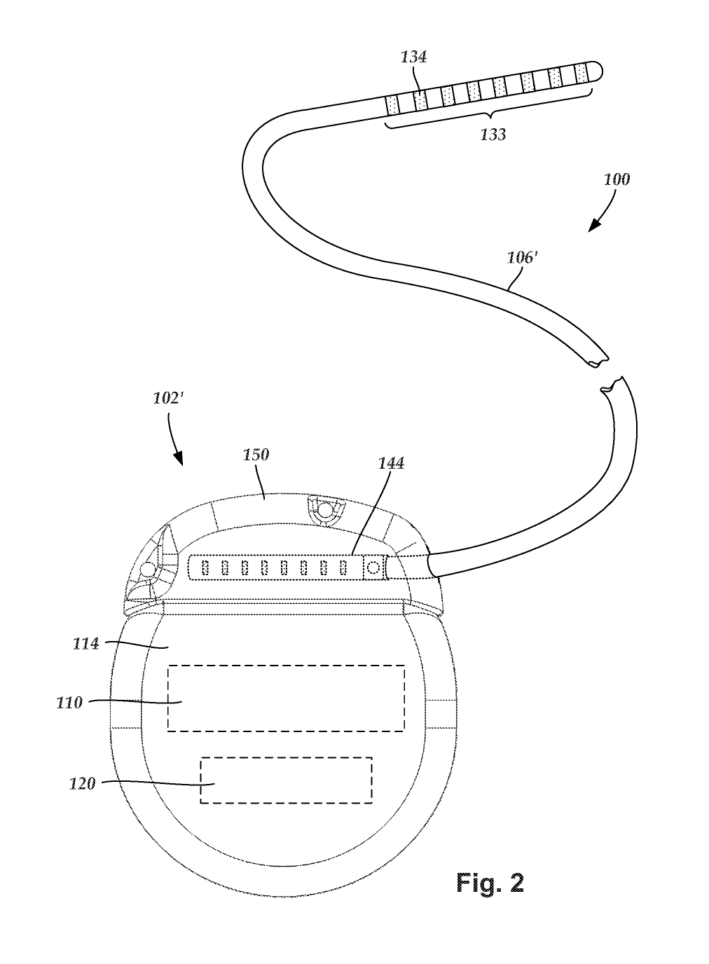

It will be understood that the electrical stimulation system can include more, fewer, or different components and can have a variety of different configurations including those configurations disclosed in the electrical stimulation system references cited herein. For example, instead of a paddle body 104, the electrodes 134 can be disposed in an array at or near the distal end of a lead body 106′ forming a percutaneous lead, as illustrated in FIG. 2. The percutaneous lead may be isodiametric along the length of the lead body 106″. The lead body 106′ can be coupled with a control module 102′ with a single connector assembly 144.

The electrical stimulation system or components of the electrical stimulation system, including one or more of the lead bodies 106, the control module 102, and, in the case of a paddle lead, the paddle body 104, are typically implanted into the body of a patient. The electrical stimulation system can be used for a variety of applications including, but not limited to, spinal cord stimulation, brain stimulation, neural stimulation, muscle activation via stimulation of nerves innervating muscle, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

The number of electrodes 134 in the array of electrodes 133 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used. In FIG. 1, sixteen electrodes 134 are shown. The electrodes 134 can be formed in any suitable shape including, for example, round, oval, triangular, rectangular, pentagonal, hexagonal, heptagonal, octagonal, or the like.

The electrodes of the paddle body 104 or one or more lead bodies 106 are typically disposed in, or separated by, a non-conductive, biocompatible material including, for example, silicone, polyurethane, and the like or combinations thereof. The paddle body 104 and one or more lead bodies 106 may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachably coupled together.

Terminals (e.g., 310 in FIG. 3A) are typically disposed at the proximal end of the one or more lead bodies 106 for connection to corresponding conductive contacts (e.g., 316 in FIG. 3A) in connector assemblies (e.g., 144 in FIG. 1) disposed on, for example, the control module 102 (or to other devices, such as conductive contacts on a lead extension, an operating room cable, a splitter, an adaptor, or the like).

Conductive wires (not shown) extend from the terminals (e.g., 310 in FIG. 3A) to the electrodes 134. Typically, one or more electrodes 134 are electrically coupled to a terminal (e.g., 310 in FIG. 3A). In some embodiments, each terminal (e.g., 310 in FIG. 3A) is only coupled to one electrode 134.

The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet wire to facilitate placement of the lead within a body of a patient. Additionally, there may also be one or more lumens (not shown) that open at, or near, the distal end of the lead, for example, for infusion of drugs or medication into the site of implantation of the paddle body 104. The one or more lumens may, optionally, be flushed continually, or on a regular basis, with saline, epidural fluid, or the like. The one or more lumens can be permanently or removably sealable at the distal end.

As discussed above, the one or more lead bodies 106 may be coupled to the one or more connector assemblies 144 disposed on the control module 102. The control module 102 can include any suitable number of connector assemblies 144 including, for example, two three, four, five, six, seven, eight, or more connector assemblies 144. It will be understood that other numbers of connector assemblies 144 may be used instead. In FIG. 1, each of the two lead bodies 106 includes eight terminals that are shown coupled with eight conductive contacts disposed in a different one of two different connector assemblies 144.

FIG. 3A is a schematic side view of one embodiment of a plurality of connector assemblies 144 disposed on the control module 102. In at least some embodiments, the control module 102 includes two connector assemblies 144. In at least some embodiments, the control module 102 includes four connector assemblies 144. In FIG. 3A, proximal ends 306 of the plurality of lead bodies 106 are shown configured and arranged for insertion to the control module 102. FIG. 3B is a schematic side view of one embodiment of a single connector assembly 144 disposed on the control module 102′. In FIG. 3B, the proximal end 306 of the single lead body 106′ is shown configured and arranged for insertion to the control module 102′.

In FIGS. 3A and 3B, the one or more connector assemblies 144 are disposed in the header 150. In at least some embodiments, the header 150 defines one or more ports 304 into which the proximal end(s) 306 of the one or more lead bodies 106/106′ with terminals 310 can be inserted, as shown by directional arrows 312, in order to gain access to the connector contacts disposed in the one or more connector assemblies 144.

The one or more connector assemblies 144 each include a connector housing 314 and a plurality of connector contacts 316 disposed therein. Typically, the connector housing 314 defines a port (not shown) that provides access to the plurality of connector contacts 316. In at least some embodiments, one or more of the connector assemblies 144 further includes a retaining element 318 configured and arranged to fasten the corresponding lead body 106/106′ to the connector assembly 144 when the lead body 106/106′ is inserted into the connector assembly 144 to prevent undesired detachment of the lead body 106/106′ from the connector assembly 144. For example, the retaining element 318 may include an aperture through which a fastener (e.g., a set screw, pin, or the like) may be inserted and secured against an inserted lead body 106/106′.

When the one or more lead bodies 106/106′ are inserted into the one or more ports 304, the connector contacts 316 can be aligned with the terminals 310 disposed on the one or more lead bodies 106/106′ to electrically couple the control module 102 to the electrodes (134 of FIG. 1) disposed at a distal end of the one or more lead bodies 106. Examples of connector assemblies in control modules are found in, for example, U.S. Pat. No. 7,244,150 and U.S. patent application Ser. No. 11/532,844, which are incorporated by reference.

In at least some embodiments, the electrical stimulation system includes one or more lead extensions. The one or more lead bodies 106/106′ can be coupled to one or more lead extensions which, in turn, are coupled to the control module 102/102′. In FIG. 3C, a lead extension connector assembly 322 is disposed on a lead extension 324. The lead extension connector assembly 322 is shown disposed at a distal end 326 of the lead extension 324. The lead extension connector assembly 322 includes a contact housing 328. The contact housing 328 defines at least one port 330 into which a proximal end 306 of the lead body 106′ with terminals 310 can be inserted, as shown by directional arrow 338. The lead extension connector assembly 322 also includes a plurality of connector contacts 340. When the lead body 106′ is inserted into the port 330, the connector contacts 340 disposed in the contact housing 328 can be aligned with the terminals 310 on the lead body 106 to electrically couple the lead extension 324 to the electrodes (134 of FIG. 1) disposed at a distal end (not shown) of the lead body 106′.

The proximal end of a lead extension can be similarly configured and arranged as a proximal end of a lead body. The lead extension 324 may include a plurality of conductive wires (not shown) that electrically couple the connector contacts 340 to terminal on a proximal end 348 of the lead extension 324. The conductive wires disposed in the lead extension 324 can be electrically coupled to a plurality of terminals (not shown) disposed on the proximal end 348 of the lead extension 324. In at least some embodiments, the proximal end 348 of the lead extension 324 is configured and arranged for insertion into a lead extension connector assembly disposed in another lead extension. In other embodiments (as shown in FIG. 3C), the proximal end 348 of the lead extension 324 is configured and arranged for insertion into the connector assembly 144 disposed on the control module 102′.

It will be understood that the control modules 102/102′ can receive either lead bodies 106/106′ or lead extensions 324. It will also be understood that the electrical stimulation system 100 can include a plurality of lead extensions 224. For example, each of the lead bodies 106 shown in FIGS. 1 and 3A can, alternatively, be coupled to a different lead extension 224 which, in turn, are each coupled to different ports of a two-port control module, such as the control module 102 of FIGS. 1 and 3A.

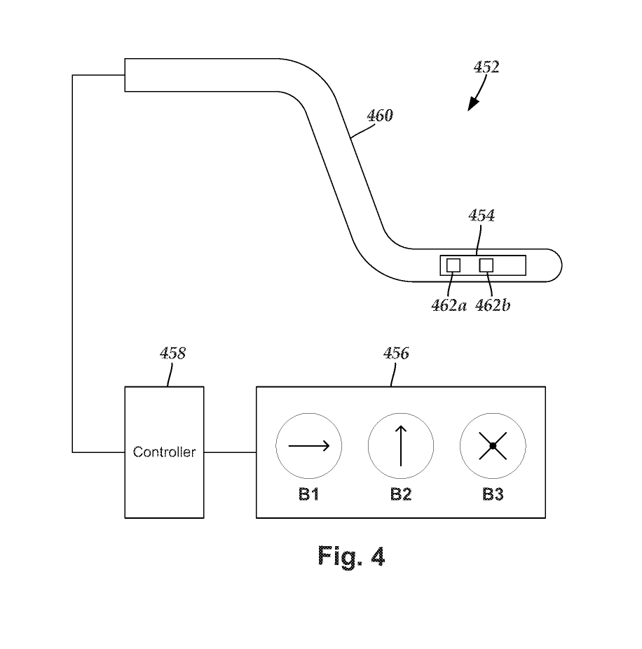

Turning to FIG. 4, placement of electrodes at a target stimulation location within patient, for example along one or more spinal cord levels within the patient's epidural space, can be important for therapy. At least some lead placement techniques involve using an introducer, such as an epidural needle, to place an electrode-containing (e.g., distal) portion of a lead in proximity to the target stimulation location. Once the lead is placed, the introducer can be removed, leaving the lead at, or in proximity to, the target stimulation location. One or more stylets may, optionally, be inserted into the lead provide sufficient rigidity to enable further positioning of the electrodes within the patient, as needed.

It is often desirable to insert medical devices, such as leads, stylets, and introducers, into regions of a patient's body with size constraints that restrict the sizes of devices able to enter these regions. Unfortunately, such size constraints (particularly along axes transverse to longitudinal lengths of the medical devices) prevent the placement of conventional sensors along some medical devices (e.g., leads, stylets, and introducers).

Placement of elongated medical devices within patients is often facilitated by one or more imaging techniques. For example, at least some conventional techniques for placement of the electrode-containing portions of an electrical stimulation lead involves using fluoroscopy for navigational guidance of one or more of the lead, introducer, or stylet. Fluoroscopy, however, can be cumbersome, expensive, and expose the patient and medical practitioners to undesired levels of potentially-harmful radiation.

As herein described, an electrical stimulation system includes a tracking system for facilitating placement of an electrical stimulation lead at a target stimulation location within a patient by tracking the positioning and orientation of a sensor assembly positioned at a location that is in proximity to lead electrodes and that is fixed with respect to the electrodes during lead placement. In some embodiments, the sensor assembly is disposed on or in the lead. In some embodiments, the sensor assembly is disposed on or in one of an introducer or a stylet used during lead placement in lieu of, or in addition to, the lead.

FIG. 4 is a diagram illustrating a tracking system 452 including a sensor assembly 454, magnetic field generator 456, a controller 458, and an elongated device 460 (e.g., a lead, a stylet, an introducer). Examples of tracking systems, sensor assemblies, magnetic field generators, and controllers suitable for use with elongated medical devices are found in, for example, U.S. patent application Ser. No. 15/384,230; and U.S. Provisional Patent Application Ser. Nos. 62/374,559; 62/455,339; 62/455,316; 62/455,299; 62/436,991; 62/436,422; and 62/436,418, all of which are incorporated by reference.

The sensor assembly 454 can be positioned within the elongated device 460, for example, at a distal end of the elongated device 460. The tracking system 452 is configured to determine the location and orientation of the sensor assembly 454 and, therefore, the elongated device 460. Magnetic fields generated by the magnetic field generator 456 provide a frame of reference for the tracking system 452 such that the location and orientation of the sensor assembly 454 is determined relative to the generated magnetic fields. The tracking system 452 can be used in a medical procedure, where the elongated device 460 is inserted into a patient and the sensor assembly 454 is used to assist with tracking the location of the elongated device 460 in the patient.

The sensor assembly 454 is communicatively coupled to the controller 458 by a wired or wireless communications path such that the controller 458 sends and receives various signals to and from the sensor assembly 454. The magnetic field generator 456 is configured to generate one or more magnetic fields. For example, the magnetic field generator 456 is configured to generate at least three magnetic fields B1, B2, and B3. Each of the magnetic fields B1, B2, and B3 is directed in a different direction, as indicated by arrows in FIG. 4. Magnetic field B1 is a magnetic field in the horizontal direction, magnetic field B2 is a magnetic field in the vertical direction, and magnetic field B3 is a magnetic field into the page of FIG. 4. The controller 458 is configured to control the magnetic field generator 104 via a wired or wireless communications path to generate one or more of the magnetic fields B1, B2, and B3 to assist with tracking the sensor assembly 454 (and therefore elongated device 460).

The sensor assembly 454 is configured to sense the generated magnetic fields and provide tracking signals indicating the location and orientation of the sensor assembly 454 in up to six degrees of freedom (i.e., x, y, and z measurements, and pitch, yaw, and roll angles). Generally, the number of degrees of freedom that a tracking system is able to track depends on the number of magnetic field sensors and magnetic field generators. For example, a tracking system with a single magnetic field sensor may not be capable of tracking roll angles and thus are limited to tracking in only five degrees of freedom (i.e., x, y, and z coordinates, and pitch and yaw angles). This is because a magnetic field sensed by a single magnetic field sensor does not change as the single magnetic field sensor is “rolled.” As such, the sensor assembly 454 includes at least two magnetic field sensors, 11OA and 11OB. The magnetic field sensors can include sensors such as inductive sensing coils and/or various sensing elements such as magneto-resistive (MR) sensing elements (e.g., anisotropic magneto-resistive (AMR) sensing elements, giant magneto-resistive (GMR) sensing elements, tunneling magneto-resistive (TMR) sensing elements, Hall effect sensing elements, colossal magneto-resistive (CMR) sensing elements, extraordinary magneto-resistive (EMR) sensing elements, spin Hall sensing elements, and the like), giant magneto-impedance (GMI) sensing elements, and/or flux-gate sensing elements. In addition, the sensor assembly 454 and/or the elongated device 460 can feature other types of sensors, such as temperature sensors, ultrasound sensors, or the like.

The sensor assembly 454 is configured to sense each of the magnetic fields B1, B2, and B3 and provide signals to the controller 458 that correspond to each of the sensed magnetic fields B1, B2, and B3. The controller 458 receives the signals from the sensor assembly 454 via the communications path and determines the position and orientation of the sensor assembly 454 and elongated device 460 in relation to the generated magnetic fields B1, B2, and B3.

The magnetic field sensors, 462A and 462B, can be powered by voltages or currents to drive or excite elements of the magnetic field sensors. The magnetic field sensor elements receive the voltage or current and, in response to one or more of the generated magnetic fields, the magnetic field sensor elements generate sensing signals, which are transmitted to the controller 458. The controller 458 is configured to control the amount of voltage or current to the magnetic field sensors and to control the magnetic field generators 456 to generate one or more of the magnetic fields B1, B2, and B3. The controller 458 is further configured to receive the sensing signals from the magnetic field sensors and to determine the location and orientation of the sensor assembly 454 (and therefore elongated device 460) in relation to the magnetic fields B1, B2, and B3.

The controller 458 can be implemented using firmware, integrated circuits, and/or software modules that interact with each other or are combined together. For example, the controller 458 may include computer-readable instructions/code for execution by a processor. Such instructions may be stored on a non-transitory computer-readable medium and transferred to the processor for execution. In general, the controller 458 can be implemented in any form of circuitry suitable for controlling and processing magnetic tracking signals and information.

In at least some embodiments, the magnetic field generator and processing unit for the magnetic sensor may operate independently from the electrical stimulator, so that the positional detection is separate from stimulation activities. In other embodiments, the magnetic field generator systems interphases (via electrical connection or wirelessly) with the electrical stimulator to determine the position of the sensor. In at least some embodiments, the electrical stimulator and the magnetic sensor communicate with each other to transfer information from the applied fields and the readings from the sensor that can be used to determine the sensor position. In some embodiments, the magnetic field generator and electrical stimulator communicate (via wire or wireless interphases) with a third system that uses the information to determine the sensor location.

In some embodiments, the sensor location is presented (e.g. displayed or described) relative to the external magnetic field positional reference. In some embodiments, the location is displayed relative to anatomical structures acquired via anatomical atlas or imaging from the patient (e.g. X-ray, fluoroscopic images, CT-scans, MM, PET scans, ultrasound, or the like). In some embodiments, the location of the sensor is described relative to other sensor information (e.g. other magnetic sensors), or other sensed signals from the body (e.g. estimates of one or more structures that generate electrical activity, for example, electrically evoked compound action potentials (eCAPs), local field potentials (LFPs), or the like, or electrical activity that is measurable, for example, via EEG, EKG, or the like).

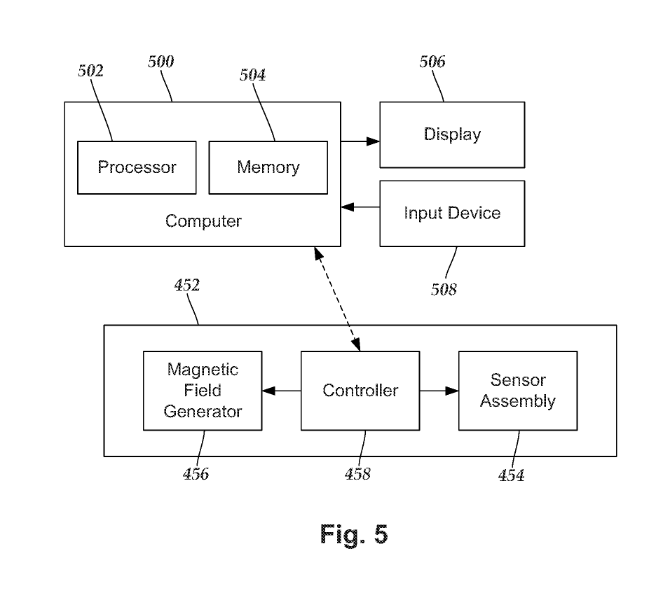

FIG. 5 illustrates one embodiment of a system for practicing the invention. The system can include a computer 500 or any other similar device that includes a processor 502 and a memory 504, a display 506, an input device 508, and the tracking system 452.

The computer 500 can be a laptop computer, desktop computer, tablet, mobile device, smartphone or other devices that can run applications or programs, or any other suitable device for processing information and for presenting a user interface. The computer 500 can be local to the user or can include components that are non-local to the user including one or both of the processor 502 or memory 504 (or portions thereof). For example, in some embodiments, the user may operate a terminal that is connected to a non-local computer. In other embodiments, the memory can be non-local to the user.

The computer 500 can utilize any suitable processor 502 including one or more hardware processors that may be local to the user or non-local to the user or other components of the computer. The processor 502 is configured to execute instructions provided to the processor, as described below.

Any suitable memory 504 can be used for the processor 502. The memory 504 illustrates a type of computer-readable media, namely computer-readable storage media. Computer-readable storage media may include, but is not limited to, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computer.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The display 506 can be any suitable display device, such as a monitor, screen, display, or the like, and can include a printer. The input device 508 can be, for example, a keyboard, mouse, touch screen, track ball, joystick, voice recognition system, or any combination thereof, or the like and can be used by the user to interact with a user interface or clinical effects map.

The tracking system 452 can include, for example, the sensor assembly 454 (disposed in an elongated medical device), the magnetic field generator 456 (disposed, for example, in an operating room table upon which a lead placement procedure is performed), and the controller 452. The tracking system 452, via the controller 452 may communicate with the computer 500 through a wired or wireless connection or, alternatively or additionally, a user can provide information between the tracking system 452 and the computer 500 using a computer-readable medium or by some other mechanism. In some embodiments, the computer 500 may include part of the tracking system 452.

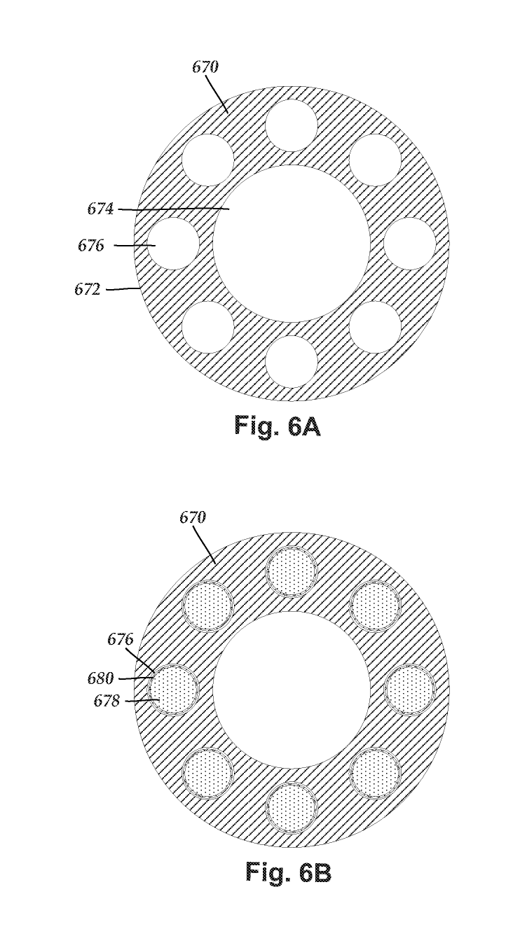

Turning to FIGS. 6A-8B, in at least some embodiments the sensor assembly is disposed along an electrical stimulation lead. Electrical stimulation leads include bodies that may define one or more lead lumens. The lead lumens can be used for a number of different functions including, for example, receiving conductive wires/cables (conductors) extending between terminals and electrodes, receiving a stylet for facilitating lead placement, delivering drugs, or the like. In at least some embodiments, the sensor assembly is disposed along a portion of one or more of the lead lumens.

FIG. 6A shows, in transverse cross-sectional view, one embodiment of a lead body 670 with an outer surface 672. In at least some embodiments, one or more layers of material are disposed over the outer surface 672. In the illustrated embodiment, the lead body 670 defines a central lumen 674 suitable for receiving a stylet and eight conductor lumens, such as conductor lumen 676, suitable for receiving conductors. Although not shown in FIG. 6A (or other figures), other lead lumens can be defined along the lead body 670 in addition to, or in lieu of, the central lumen and conductor lumens.

In some embodiments, the lead body defines no central lumen or multiple central lumens 674. The lead body 670 can define any suitable number of conductor lumens 676 (e.g., one, two, three, four, five, six, seven, eight, nine, ten, eleven, twelve, thirteen, fourteen, fifteen, sixteen, or more conductor lumens). The number of conductors utilized can, in some embodiments, be based, in part, on the number of electrodes, the number of conductors disposed in the conductor lumens, or both, or other factor, or combination of factors.

FIG. 6B shows, in transverse cross-sectional view, one embodiment of the lead body 670 with a conductor, such as conductor 678, disposed in each of the conductor lumens 676. In some embodiments, at least one of the conductors is encased in electrically nonconductive material 680. In FIG. 6B, a single conductor is shown disposed in each of the conductor lumens 676. In alternate embodiments, at least one of the conductor lumens is configured to receive multiple conductors.

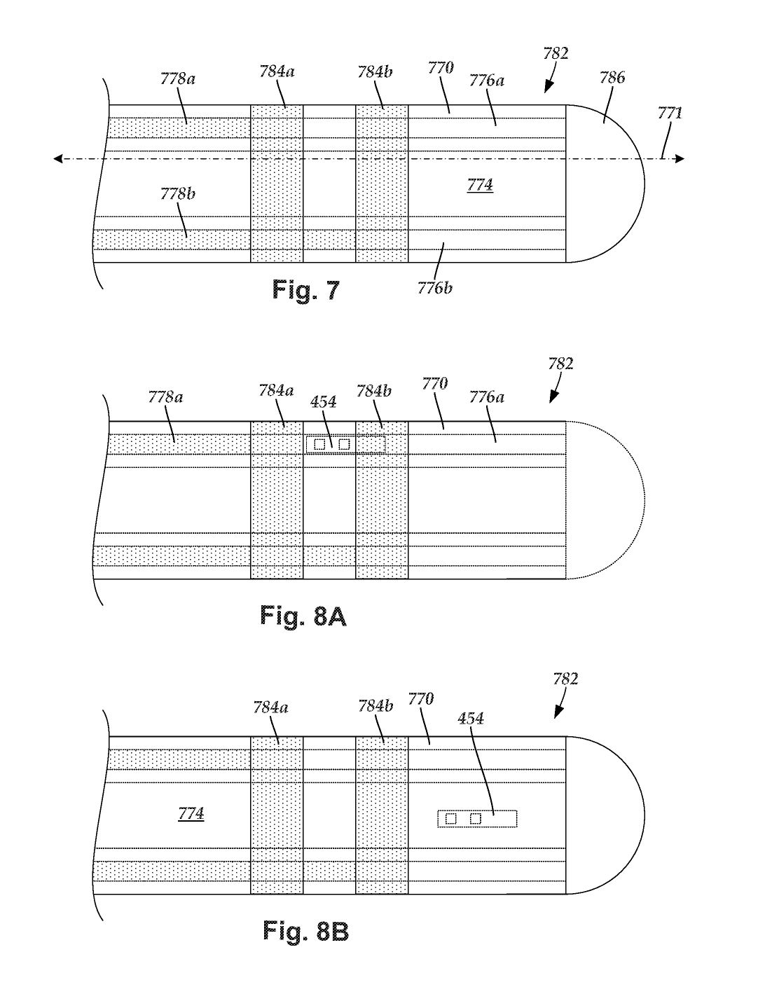

FIG. 7 shows, in side view, one embodiment of a distal portion of a lead 782. The lead 782 includes a lead body 770 having a longitudinal length 771. A distal tip 786 is disposed at a distal-most portion of the lead body 770. Two electrodes 784a, 784b are disposed along a lead body 770, with the electrode 784a being disposed more proximally along a longitudinal length 771 of the lead body 770 from the electrode 784b. In other words, the electrode 784a is more distant from the distal tip 786 than the electrode 784b. A central lumen 774 and two conductor lumens 776a, 776b are shown defined in the lead body 770. Conductors 778a, 778b extend along the lead body 770 within the conductor lumens 776a, 776b, respectively, and electrically couple to the electrodes 784a, 784b, respectively. The number of electrodes and conductor lumens shown in FIG. 5 is for clarity of invention. As described above, the lead can include other numbers of electrodes and lead lumens.

As shown in FIG. 7, the conductor lumens and the central lumen may extend distally from the electrodes. In at least some embodiments, the conductor lumens and the central lumen extend to the distal tip 786. In the case of the conductor lumens, once the conductor(s) within the conductor lumen are coupled to their corresponding electrode(s), the conductor lumens may include an empty region extending distally long the lead body from the electrode(s) to which the conductor is/are attached. In some instances, epoxy is inserted into the conductor lumens to fill the empty space within the conductor lumens distal to the coupled electrode(s).

Turning to FIGS. 8A-8B, in at least some embodiments, the sensor assembly is disposed in one of the lead lumens. In at least some embodiments, the sensor assembly is in proximity to at least one of the electrodes. In at least some embodiments, the sensor assembly at least partially overlaps with at least one of the electrodes along the longitudinal length of the lead body. In at least some embodiments, the sensor assembly is entirely distal to at least one of the electrodes along the longitudinal length of the lead body. In at least some embodiments, the sensor assembly is entirely distal to each of the electrodes along the longitudinal length of the lead body.

The sensor assembly includes longitudinal dimensions and transverse dimensions that are orthogonal to the longitudinal dimensions. In at least some embodiments, the sensor assembly is disposed in the electrical stimulation lead with the longitudinal dimensions of the sensor array extending along the long axis of the electrical stimulation lead locally to the sensor assembly. Accordingly, the transverse dimensions of the sensor array are locally orthogonal to the long axis of the electrical stimulation lead.

In at least some embodiments, the sensor assembly 454 has a largest transverse dimension that is no greater than 2 mm, 1.5 mm, 1 mm, 0.9 mm, 0.8 mm, 0.7 mm, 0.6 mm, 0.5 mm, 0.4 mm, 0.3 mm, 0.2 mm, 0.1 mm. In at least some embodiments, the sensor assembly 454 has a largest transverse dimension that is no greater than 1 mm and no less than 0.2 mm. In at least some embodiments, the sensor assembly 454 has a largest transverse dimension that is no greater than 0.5 mm and no less than 0.1 mm. It will be understood that, in FIGS. 8A-8B and in other figures, the relative size of the sensor assembly to the devices (e.g., the electrical stimulation lead, stylet, lead introducer) within which the sensor assembly is shown disposed in, may be altered for clarity of illustration, and is not meant to be limiting.

FIG. 8A shows, in side view, one embodiment of the sensor assembly 454 disposed in the conductor lumen 776a of the lead 782. In at least some embodiments, the sensor assembly 454 is disposed in the conductor lumen 776a distally from the conductor 778a, which is also disposed in the conductor lumen 776, and the electrode 784a to which the conductor 778a is coupled. When the sensor assembly is disposed in one of the conductor lumens, it may be advantageous to position the sensor assembly distally from any conductors also disposed in the conductor lumen, as well as any electrodes to which the conductor(s) is/are coupled to provide additional space in the conductor lumen.

In at least some embodiments, the sensor assembly is disposed in a conductor lumen that houses a conductor that couples with one of the proximal-most electrodes along the longitudinal length 771 of the lead body 770. In at least some embodiments, the sensor assembly is disposed in a conductor lumen that houses the conductor that couples with the proximal-most electrode along the longitudinal length 771 of the lead body 770. It may be advantageous to dispose the sensor assembly in a conductor lumen that houses a conductor that couples with one of the proximal-most electrodes along the longitudinal length 771 of the lead body 770. Such a configuration increases the length of the open space available to house the sensor assembly from the remaining conductor lumens. Additionally, such a configuration may enable the sensor assembly to better align with the electrodes along the longitudinal length 771 of the lead body 770 than conductor lumens housing conductors coupled to more distally positioned electrodes. In at least some embodiments, the sensor assembly is disposed in a lead lumen that does not house any conductors.

FIG. 8B shows, in side view, one embodiment of the sensor assembly 454 disposed in the central lumen 774 of the lead 782. In at least some embodiments, the sensor assembly 454 is disposed in the central lumen 774 at the same location along the longitudinal length 771 of the lead body 770 as the electrodes 784a, 784b. In at least some embodiments, the sensor assembly 454 is disposed in the central lumen 774 distally from the electrodes 784a, 784b along the longitudinal length 771 of the lead body 770.

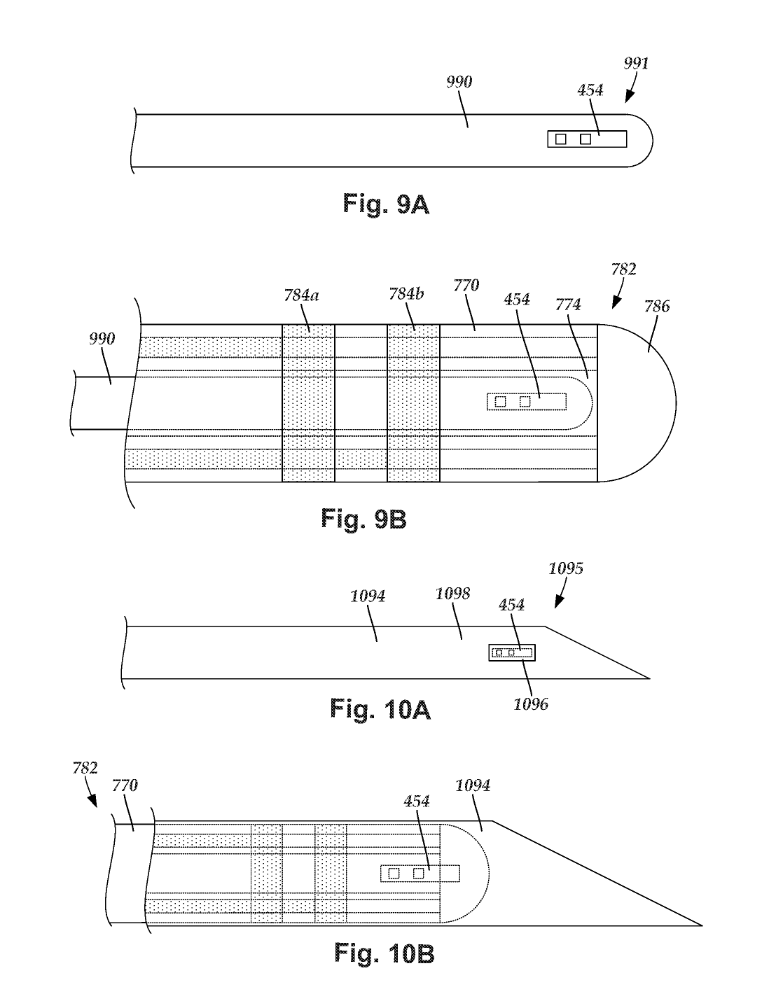

Turning to FIGS. 9A-9B, in at least some embodiments the sensor assembly is disposed on or in a stylet suitable for facilitating placement of an electrical stimulation lead into a patient. FIG. 9A shows, in side view, one embodiment of a distal portion of a stylet 990 suitable for insertion into an electrical stimulation lead to facilitate placement of the lead in a patient. The sensor assembly 454 is disposed on or in the distal portion of the stylet 990 at a location suitable for aligning with, or approximately aligning with, electrodes of the lead when the stylet 990 is inserted into the lead.

FIG. 9B shows, in side view, the distal portion of the stylet 990 disposed in the central lumen 774 of the lead 782. The placement of the sensor assembly 454 along the stylet 990 can, optionally, be adjusted so that the sensor assembly 454 is even with, or in proximity to, the electrodes 784a, 784b along the longitudinal length 771 of the lead body 770 during a lead placement procedure. The placement of the sensor assembly 454 can adjusted based on the positioning of the sensor assembly 454 along the stylet 990, or adjusting how far into the central lumen 774 the stylet extends. For example, an end stop can, optionally, be disposed in the central lumen 774 in proximity to the distal tip 786 to adjust how far into the central lumen 774 the stylet extends. In at least some embodiments, the sensor assembly 454 is disposed at a distal tip 991 of the stylet 990.

Turning to FIGS. 10A-10B, in at least some embodiments, the sensor assembly is disposed on or in a lead introducer (e.g., an epidural needle or cannula) suitable for placing an electrical stimulation lead within a patient. FIG. 10A shows, in side view, one embodiment of a distal portion of a needle 1094 suitable for receiving an electrical stimulation lead to facilitate placement of the lead in a patient. The sensor assembly 454 is disposed on or in a distal portion 1095 of the needle 1094 at a location suitable for aligning, or approximately aligning, with electrodes of the lead when the needle 1094 receives the lead. In at least some embodiments, the sensor assembly 454 is disposed along a cutout 1096 along a wall 1098 of the needle 1094.

FIG. 10B shows, in side view, the distal portion of the lead 782 disposed in the distal portion of the needle 1094. The placement of the sensor assembly 454 along the needle 1094 can, optionally, be adjusted so that the sensor assembly 454 is even with, or in proximity to, the electrodes 784a, 784b along the longitudinal length 771 of the lead body 770 when the lead 782 is received by the needle 1094. The placement of the sensor assembly 454 can adjusted based on the positioning of the sensor assembly 454 along the needle 1094, or adjusting how far into the needle 1094 the lead 782 extends during placement of the lead 782.

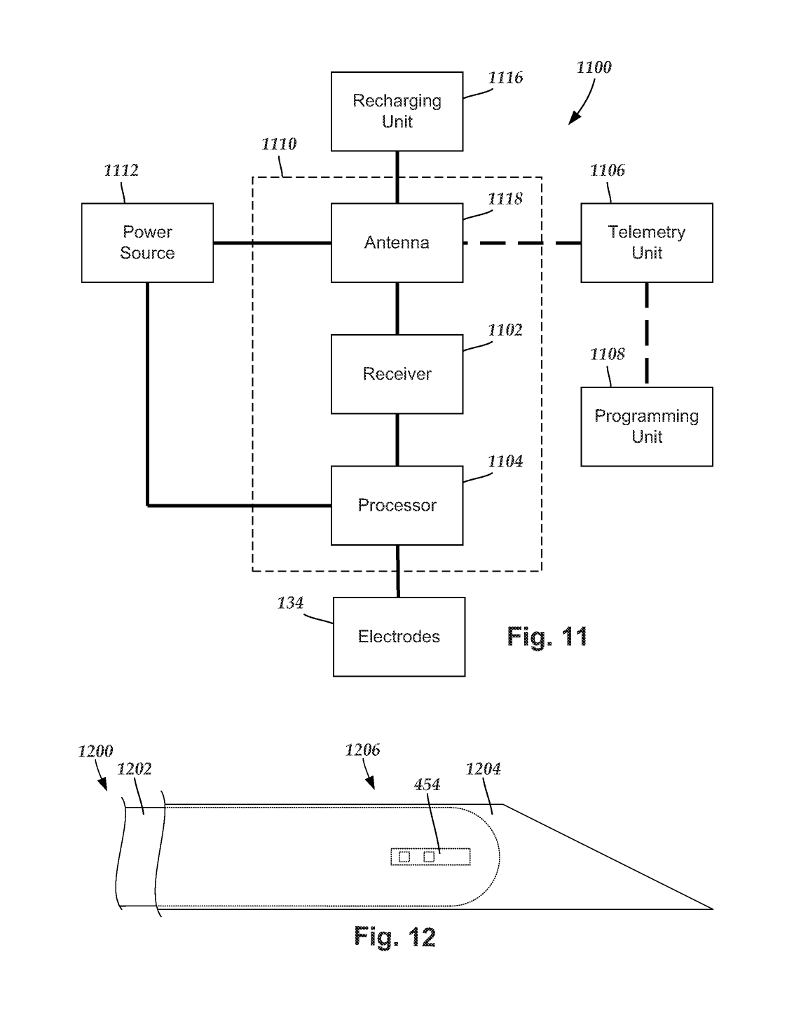

FIG. 11 is a schematic overview of one embodiment of components of an electrical stimulation system 1100 including an electronic subassembly 1110. It will be understood that the electrical stimulation system can include more, fewer, or different components and can have a variety of different configurations including those configurations disclosed in the stimulator references cited herein.

Some of the components (for example, a power source 1112, an antenna 1118, a receiver 1102, and a processor 1104) of the electrical stimulation system can be positioned on one or more circuit boards or similar carriers within a sealed housing of an implantable pulse generator (see e.g., control module 102 in FIG. 1), if desired. Any power source 1112 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1118 or a secondary antenna. The external power source can be in a device that is mounted on the skin of the user or in a unit that is provided near the user on a permanent or periodic basis.

If the power source 1112 is a rechargeable battery, the battery may be recharged using the optional antenna 1118, if desired. Power can be provided to the battery for recharging by inductively coupling the battery through the antenna to a recharging unit 316 external to the user. Examples of such arrangements can be found in the references identified above. The electronic subassembly 1110 and, optionally, the power source 1112 can be disposed within a control module (e.g., 102 of FIG. 1).

In one embodiment, electrical stimulation signals are emitted by the electrodes 134 on the paddle or lead body to stimulate nerve fibers, muscle fibers, or other body tissues near the electrical stimulation system. The processor 1104 is generally included to control the timing and electrical characteristics of the electrical stimulation system. For example, the processor 1104 can, if desired, control one or more of the timing, frequency, strength, duration, and waveform of the pulses. In addition, the processor 1104 can select which electrodes can be used to provide stimulation, if desired. In some embodiments, the processor 1104 selects which electrode(s) are cathodes and which electrode(s) are anodes. In some embodiments, the processor 1104 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 308 that, for example, allows modification of pulse characteristics. In the illustrated embodiment, the processor 1104 is coupled to a receiver 1102 which, in turn, is coupled to the optional antenna 1118. This allows the processor 1104 to receive instructions from an external source to, for example, direct the pulse characteristics and the selection of electrodes, if desired.

In one embodiment, the antenna 1118 is capable of receiving signals (e.g., RF signals) from an external telemetry unit 1106 which is programmed by the programming unit 1108. The programming unit 1108 can be external to, or part of, the telemetry unit 1106. The telemetry unit 1106 can be a device that is worn on the skin of the user or can be carried by the user and can have a form similar to a pager, cellular phone, or remote control, if desired. As another alternative, the telemetry unit 1106 may not be worn or carried by the user but may only be available at a home station or at a clinician's office. The programming unit 1108 can be any unit that can provide information to the telemetry unit 1106 for transmission to the electrical stimulation system 1100. The programming unit 1108 can be part of the telemetry unit 1106 or can provide signals or information to the telemetry unit 1106 via a wireless or wired connection. One example of a suitable programming unit is a computer operated by the user or clinician to send signals to the telemetry unit 1106.

The signals sent to the processor 1104 via the antenna 1118 and the receiver 1102 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1100 to cease operation, to start operation, to start charging the battery, or to stop charging the battery. In other embodiments, the stimulation system does not include the antenna 1118 or receiver 1102 and the processor 1104 operates as programmed.

Optionally, the electrical stimulation system 1100 may include a transmitter (not shown) coupled to the processor 1104 and the antenna 1118 for transmitting signals back to the telemetry unit 1106 or another unit capable of receiving the signals. For example, the electrical stimulation system 1100 may transmit signals indicating whether the electrical stimulation system 1100 is operating properly or not or indicating when the battery needs to be charged or the level of charge remaining in the battery. The processor 1104 may also be capable of transmitting information about the pulse characteristics so that a user or clinician can determine or verify the characteristics.

Turning to FIG. 12, the electromagnetic tracking system described above can also be used to facilitate placement of a radiofrequency ablation system within a patient. Examples of radiofrequency ablation systems with thermocouple electrodes and cannulas and, optionally, stylets are found in, for example, U.S. Patent Applications Publication Nos. 2017/0050038; 2017/0049993; 2017/0056093; 2017/0065342; 2017/0049503; 2017/0050017; 2017/0049507; 2017/0050041; 2017/0049513, all of which are incorporated by reference.

FIG. 12 shows, in side view, a radiofrequency ablation system 1200 that includes a thermocouple electrode 1202 inserted into a cannula 1204. The sensor assembly 454 can be used to facilitate introduction of the thermocouple electrode 1202 into a patient, as described above. The sensor assembly 454 can be disposed along a shaft of the thermocouple electrode 1202. In at least some embodiments, the sensor assembly 454 is disposed along a distal portion 1206 of the thermocouple electrode 1202. Additionally, or alternately, the sensor assembly 454 can be disposed along the cannula (e.g., along a cutout in a wall of the cannula, as shown in FIG. 10A) 1204, as described above with respect to introducers and stylets.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation lead comprising
a lead body having a proximal portion, a distal portion, and a longitudinal length,
a plurality of electrodes disposed along the distal portion of the lead body,
a plurality of terminals disposed along the proximal portion of the lead body, and
a plurality of conductors electrically coupling the plurality of terminals to the plurality of electrodes;
a sensor assembly disposed in the lead body in proximity to the plurality of electrodes, the sensor assembly configured and arranged for sensing a magnetic field in three different directions to facilitate determination of a position and orientation of the plurality of electrodes based on the sensing, wherein the sensor assembly has a largest transverse dimension of no greater than 0.5 millimeters.

2. The electrical stimulation lead of claim 1, wherein the sensor assembly is aligned with at least one electrode of the plurality of electrodes along the longitudinal length of the lead body.

3. The electrical stimulation lead of claim 1, wherein the electrical stimulation lead further comprises a distal tip, and wherein the sensor assembly is disposed along the distal portion of the lead between the plurality of electrodes and the distal tip.

4. The electrical stimulation lead of claim 1, wherein the lead body defines a central lumen extending along the longitudinal length of the lead body, and wherein the sensor assembly is disposed in the central lumen.

5. The electrical stimulation lead of claim 1, wherein the lead body defines a plurality of conductor lumens extending along the longitudinal length of the lead body, wherein the conductors are disposed in the conductor lumens, and wherein the sensor assembly is also disposed in one of the conductor lumens.

6. The electrical stimulation lead of claim 5, wherein the sensor assembly is disposed in one of the conductor lumens distally from the at least one conductor of the plurality of conductors which is also disposed in that conductor lumen.

7. The electrical stimulation lead of claim 5,
wherein the plurality of electrodes comprises a proximal-most electrode along the longitudinal length of the lead body,
wherein the plurality of conductors comprises a first conductor,
wherein the plurality of conductor lumens comprises a first conductor lumen,
wherein the first conductor is disposed in the first conductor lumen and is electrically coupled to the proximal-most electrode,
wherein the sensor assembly is disposed in the first conductor lumen distally from the first conductor along the longitudinal length of the lead body.

8. An electrical stimulation system, comprising:
the electrical stimulation lead of claim 1; and
a control module coupleable to the electrical stimulation lead, the control module comprising
a housing, and
an electronic subassembly disposed in the housing.

9. The electrical stimulation system of claim 8, further comprising a controller configured and arranged to control one or more magnetic fields sensed by the sensor assembly and to determine the position and orientation of the sensor assembly with respect to the one or more magnetic fields based on received signals output from the sensor assembly.

10. The electrical stimulation system of claim 9, further comprising a magnetic field generator configured and arranged for generating the one or more magnetic fields sensed by the sensor assembly and controlled by the controller.

11. A medical device kit, comprising:
an elongated member configured and arranged for at least partially introducing into a patient;
a cannula configured and arranged to receive the elongated member and to facilitate introduction of the elongated member into the patient;
a stylet configured and arranged to insert into the elongated member during introduction of the elongated member into the patient and to facilitate introduction of the elongated member into the patient; and
a sensor assembly disposed on or in at least one of the cannula or the stylet, the sensor assembly configured and arranged for sensing a magnetic field in three different directions to facilitate determination of a position and orientation of a portion of the elongated member based on the sensing, wherein the sensor assembly has a largest transverse dimension of no greater than 0.5 millimeters.

12. The medical device kit of claim 11, wherein the elongated member is an electrical stimulation lead.

13. The medical device kit of claim 11, wherein the elongated member is a radiofrequency ablation catheter.

14. The medical device kit of any claim 11, wherein the sensor assembly is a first sensor assembly, and wherein the medical device kit comprises a second sensor assembly disposed in the elongated member.

15. A radiofrequency ablation system comprising:
a thermocouple electrode configured and arranged for insertion into a patient and for conducting radiofrequency current to patient tissue; and
a sensor assembly disposed along the thermocouple electrode, the sensor assembly configured and arranged for sensing a magnetic field in three different directions to facilitate determination of a position and orientation of the thermocouple electrode based on the sensing, wherein the sensor assembly has a largest transverse dimension of no greater than 0.5 millimeters.

16. A method of placing an electrical stimulation lead at a target stimulation location within a patient, the method comprising
providing the electrical stimulation lead of claim 1;
advancing the plurality of electrodes of the electrical stimulation lead to, or in proximity to, the target stimulation location; and
using the sensor assembly of the electrical stimulation lead to facilitate advancement of the plurality of electrodes to, or in proximity to, the target stimulation location.

17. The method of claim 16, wherein using the sensor assembly of the electrical stimulation lead to facilitate advancement of the plurality of electrodes comprises disposing the sensor assembly in a conductor lumen defined in the lead body of the electrical stimulation lead.

18. The method of claim 16, wherein using the sensor assembly of the electrical stimulation lead to facilitate advancement of the plurality of electrodes comprises disposing the sensor assembly in a central lumen defined in the lead body of the electrical stimulation lead.

19. The method of claim 16, wherein using the sensor assembly of the electrical stimulation lead to facilitate advancement of the plurality of electrodes comprises positioning the sensor assembly along the lead body of the electrical stimulation lead with the sensor assembly aligned with at least one electrode of the plurality of electrodes along the longitudinal length of the lead body.

20. The method of claim 16, wherein using the sensor assembly of the electrical stimulation lead to facilitate advancement of the plurality of electrodes comprises positioning the sensor assembly along the lead body of the electrical stimulation lead with the sensor assembly at least partially disposed between the plurality of electrodes and a distal tip of the electrical stimulation lead.
